(19) United States
(12) Patent Application Publication
(10) Pub. No.: US 2008/0065027 A1
(43) Pub. Date: Mar. 13, 2008

(54) Disposable Safety Syringe to Prevent Needlestick Injuries and Reuse

(76) Inventor: Fraser R. Sharp, Vancouver (CA)

Correspondence Address: NIXON & VANDERHYE, PC, 901 NORTH GLEBE ROAD, 11TH FLOOR, ARLINGTON, VA, 22203, US

(21) Appl. No.: 11/662,400

(22) PCT Filed: Sep. 15, 2005
(86) PCT No.: PCT/CA05/01411

§ 371 Date: Mar. 9, 2007

Related U.S. Application Data
(60) Provisional application No. 60/610,194, filed on Sep. 16, 2004.
(60) Provisional application No. 60/625,724, filed on Nov. 8, 2004.
(60) Provisional application No. 60/635,178, filed on Dec. 13, 2004.
(60) Provisional application No. 60/640,177, filed on Dec. 30, 2004.

(51) Int. Cl. A61M 5/315

(52) U.S. Cl. 604/220; 604/218

(57) ABSTRACT

The syringe includes a cylindrical barrel, a plunger in part disposed within the barrel and an elongated end cannula extending from the distal end of the barrel and formed integrally with the barrel of a single unitary plastic material. The plunger includes a distal end portion within the barrel having surfaces cooperable with surfaces along the interior of the barrel and enabling separation of the plunger end from remaining portions of the plunger in response to relative rotation of the plunger and barrel. A collar may be disposed about the cannula to seal with the lip of a medication vial. A clip is engageable within the plunger and barrel to fix the axial location of the plunger relative to the barrel at selected locations therealong.

### FIELD OF THE INVENTION

This application relates to prior provisional applications Ser. Nos. 60/610,194 filed Sep. 16, 2004; 60/625,724 filed Nov. 8, 2004; 60/635,178 filed Dec. 13, 2004; and 60/640,177 filed Dec. 30, 2004.

This invention relates to a novel safety disposable syringe needle system which has medical and industrial applications. More particularly, this invention pertains to a syringe which can be filled with fluid, medication or the like and which can effect transient or long term storage of medication or other substances and allow for transfer of them from the interior of the syringe into a medication vial or an intravenous line without requiring attachment to, or use of any additional devices such as detachable needles or access devices or ports. In one embodiment a conventional metal needle, Luer fit or otherwise can be attached to effect percutaneous or other injection.

The syringe tends to eliminate or reduce the likelihood of a needle stick injury. In one embodiment the syringe can be permanently disabled after use by the end user, preventing reuse of the syringe for injection or transfer of fluids. In one embodiment the material (COC) from which the syringe is made, is substantially inert, with respect to the contents allowing storage of medications or the like for prolonged periods of time without significant effects on the composition or activity.

### BACKGROUND OF THE INVENTION

Needle stick injuries among medical personnel such as healthcare workers are of a growing concern both in developed countries and in the developing world. More than 20 diseases can be transmitted through accidental needle stick injuries particularly Hepatitis B, Hepatitis C and Ebola, West Nile fever and others.

The reduction or elimination of needle stick injuries has become an important issue. Prevention of spread of disease is an important factor in a safe work place and assists recruitment and retention of healthcare workers. The “Needle Stick Safety Act” in the US and similar initiatives enacted in Canada and being discussed in Australia and Europe are forcing the adoption of safer technology for syringes and needles having built-in safety features. In developing countries the inappropriate reuse of syringes has been high. Some reports indicate that more than 50% of injections given in Africa in the recent past have been given by reuse of un-sterile and unsafe syringes. Focus has now been turned to both prevention of disease transmission to healthcare workers by prevention of accidental needlestick injuries and to patients through non reuse of syringes.

A syringe with a sharp metal needle is used for injections through the skin. The reconstitution of lyophilized or other powdered medication has traditionally been carried out using sharp metal needles to puncture the stoppers of medication vials to allow addition of diluent and reconstitutions of the drug. The use of a sharp metal needle allows inadvertent accidental needle stick injuries and/or deliberate reuse of syringes or needles for injection.

Traditionally sharp metal needles have also been used for introducing medication into Intravenous infusion lines. Attempts to eliminate needles have been made using completely blunt plastic cannulas and pre-slit septums in intravenous lines. While these have been effective, they have been complicated and expensive requiring additional valves for access to medication valves or complicated additions to the totally blunt cannula which increase expense. They have not received wide spread acceptance.

### SUMMARY OF THE INVENTION

This invention relates to a safety disposable syringe which additionally can allow for safe storage of fluids or medication within the barrel of the syringe. This enables transfer of the contained fluid into for example medication vials containing lyophilized or other drugs or substances requiring reconstitution with the fluid. The syringe can also be used for transferring the reconstituted medication into an intravenous line using the same syringe. The syringe could also be used for industrial purposes for sampling or adding substances which for example may be toxic to other vessels or containers.

A safety cannula described in U.S. Pat. No. 6,616,632, incorporated herein by reference can access medication vials and intravenous lines without the risk of needle stick injuries. In one embodiment a cannula such as described in this patent or similar could be attached to the syringe using conventional Luer fit or Luer lok connections molded as the front part of the syringe barrel. However attachment of a separate cannula is time consuming and more costly than, as in an aspect of the present invention, a syringe which has an integral safety cannula formed as a single unit with the barrel by for example injection molding.

The current invention allows for access to intravenous lines, medication vials and the like without the need for a separate cannula. In addition because of recently developed plastic resins such as cyclic olefin copolymer(COC) with general impermeability and non-reactivity, storage of dilution fluid or medication, dry or liquid, in the syringe can be achieved. This eliminates the need for a storage vial and for the commonly used sharp metal needle and syringe to access that vial. In one embodiment of the syringe, the syringe can be permanently disabled preventing reuse of the syringe or any part. This can be achieved in a number of ways including a frangible portion on the needle/cannula allowing removal of it from the syringe. In addition, a frangible portion on the plunger enables breakage of the plunger and prevents reuse of the syringe. This can be accomplished for example by fixing one portion of the plunger relative to the barrel and applying a rotational force to the unfixed portion of the plunger to cause breakage of the plunger at a predetermined break point.

It will be appreciated that the cannula, integral with the barrel may have a large variety of configurations, dimensions and surface configurations. The external side walls may be straight, smooth and cylindrical or may be tapered and smooth, both terminating in a cannula tip.

In one embodiment, the barrel and cannula are molded as one single unitary component. This simplifies manufacturing thereby reducing costs. It reduces the work needed to assemble a syringe and eliminates the need to locate, unpackage and attach any additional type of cannula or needle onto the front of the syringe. This also eliminates a step which could allow inadvertent contamination of the fluid path and an ensuing infection.

It has additional benefits such as preventing exposure of a metal needle to the medication. Metal can result in undesired interactions between the fluid within the pre-filled syringe and needle. For example one undesirable effect would be crystallization of the dissolved substance and potential blockage of the needle.

In another embodiment, the plunger can be fixed using a removable clip or similar reversible fixation mechanism in a variety of different axial positions relative to the barrel to allow accurate filling of the syringe to predetermined volumes. This allows for one syringe size to be used for different volumes of solution resulting in simplified packaging.

The axial fixation of the plunger prevents movement of the plunger relative to the barrel during transportation and/or during handling of the syringe during for example penetration of the stopper of a medication vial, or an IV line. The clip or fixation mechanism can be disengaged and removed to allow full plunger movement relative to the barrel when required.

Molding the barrel using an inert plastic material enables the contents(medication) to be exposed to as few as two materials. The possible interactions of the container and contained fluid depends on the nature of the individual materials in which it may come in contact and also the combination of materials. A greater diversity of substances can be stored within the syringe when a small number, for example, two materials are used (no metal cannula or epoxy or other adhesive materials). In one embodiment the material used does not allow for long term storage however the other features of the described syringe may be of value for shorter, transient single use.

In standard syringes the plunger tip is commonly covered by an elastomeric stopper allowing for smooth water-tight axial movement of the plunger. The delivery cannula on the end of the barrel is traditionally closed with a needle cover. In a further embodiment of the proposed syringe, both of these components, the needle cover and stopper may be of the same material. Thus a medication stored in the described syringe is exposed to only the plastic resin from which the barrel is made in addition to the stopper elastomer. Thus many medications currently stored in a glass vial or the like, with an elastomer stopper may be able to be stored in the COC plastic resin syringe.

If it is necessary to reconstitute the medication for any reason immediately prior to giving the drug, this could be accomplished by storing the unreconstituted medication within the barrel of the syringe. The cannula could then be used to draw in diluent from an external source and allow reconstitution in the syringe. Long term compatibility of the reconstituted drug with the syringe barrel material would not be necessary.

If the drug is intended to be given Intravenously, as is often used for medications, the cannula can be used to access an Intravenous port in more than one way. In addition, the syringe may also be used with a standard injection or safety needle, i.e., converted to a standard syringe with a Luer fit attached to a sharp metal needle. To accomplish this, the cannula may have a weakened portion intermediate the cannula tip and the distal end of the barrel. The weakened portion of the cannula may be external to or internal of the cannula. Additionally, the proximal portion of the cannula between the weakened portion and the distal end of the barrel may be shaped and dimensioned to receive a standard Luer fit metal needle. Also, the distal end of the barrel may be shaped and dimensioned to receive a standard Luer Lok™ needle. An externally placed but affixed component to the barrel provides such rotational and threaded function as is present in a Luer Lok. That is, the barrel may be extended beyond the base of the cannula or an external component affixed to provide an internally threaded cylindrical extension, i.e., Luer Lok™. The flanges on a standard Luer fit metal needle may then threadedly engage the internally threaded cylindrical extension to retain the Luer needle about the base of the cannula. Thus, the integrally molded syringe barrel and cannula can be converted for direct injection through an individual's skin using a standard Luer fit steel needle or a safety Luer fit needle by breaking off the distal end of the cannula. Thus the converted syringe would not be reusable once the plunger has been broken by rotation of the plunger relative to the barrel.

Following use, the syringe can be permanently disabled by breaking the plunger. The syringe cannot then be reused.

In a preferred embodiment of the present invention there is provided a syringe comprising: a syringe barrel having proximal and distal ends; a plunger in part disposed in said barrel and having a bung or stopper adjacent an end of the plunger within the barrel, said plunger being movable toward and away from the distal end of the barrel; the distal end of the barrel terminating in an elongated end cannula having a central opening in communication with the interior of the barrel between the distal end of the barrel and plunger bung for receiving fluid into or expelling fluid from the barrel; the barrel and cannula being molded from and formed of a single unitary plastic material.

In a further preferred embodiment hereof, there is provided a syringe comprising: a syringe barrel having proximal and distal ends; a plunger in part disposed in said barrel and having a bung or stopper adjacent an end of the plunger within the barrel, said plunger being movable toward and away from the distal end of the barrel; the distal end of the barrel terminating in an elongated end cannula having a central opening in communication with the interior of the barrel between the distal end of the barrel and plunger bung for receiving fluid into or expelling fluid from the barrel; the plunger having an end portion adjacent the bung and separable from remaining portions of the plunger, said plunger end portion and the barrel having cooperating surfaces enabling separation of the plunger end portion and the remaining portions of the plunger in response to relative rotation of the plunger and the barrel.

In a further preferred embodiment hereof, there is provided a syringe comprising: a syringe barrel having proximal and distal ends; a plunger in part disposed in said barrel and having a bung adjacent an end of the plunger within the barrel, said plunger being movable toward and away from the distal end of the barrel; the distal end of the barrel terminating in an elongated end cannula having a central opening in communication with the interior of the barrel between the distal end of the barrel and plunger bung for receiving fluid into or expelling fluid from the barrel; and an annular collar about the cannula, said collar having features such as an annular groove about an external surface thereof sized to receive the tip of a medication vial enabling the collar to seal between the cannula and the vial lip.

In a further preferred embodiment hereof, there is provided a syringe comprising: a syringe barrel having proximal and distal ends; a plunger in part disposed in said barrel and having a bung adjacent an end of the plunger within the barrel, said plunger being movable toward and away from the distal end of the barrel; the distal end of the barrel terminating in an elongated end cannula having a central opening in communication with the interior of the barrel between the distal end of the barrel and plunger bung for receiving fluid into or expelling fluid from the barrel; and a clip engageable with the plunger and the barrel at each of a plurality of selected axial locations of the plunger relative to the barrel thereby to fix the axial location of the plunger relative to the barrel at a selected location therealong.

### BRIEF DESCRIPTION OF THE DRAWINGS

FIG. 1 is a cross-sectional view of a syringe constructed in accordance with the preferred embodiment of the present invention;

FIGS. 1A and 1B are cross-sectional views of the syringe similar to FIG. 1 illustrating further configurations of the cannula;

FIG. 2 is a fragmentary side elevational view of an integral connection between the cannula and the syringe barrel with portions illustrated in cross-section.

FIG. 3 is a cross-sectional view of the distal ends of the barrel and plunger;

FIG. 4 is a cross-sectional view thereof taken generally about on line 4-4 in FIG. 3;

FIG. 5 is a view similar to FIG. 3 with one portion of the plunger and plunger bung parted from one another;

FIG. 6 is a view similar to FIG. 3 illustrating a collar around the cannula poised for withdrawal of fluid from a vial;

FIG. 7 is a view similar to FIG. 6 illustrating the collar attached to the vial and the distal end of the syringe and liquid withdrawn from the vial;

FIG. 8 is a view similar to FIG. 1 illustrating a clip for fixing the plunger at selected axial positions along the barrel;

FIG. 9 is a plan view of the clip;

FIG. 10 is a side elevational view of the clip;

FIG. 11 is a side elevational view similar to FIG. 8 illustrating a further form of axial fixation of the plunger relative to the barrel;

FIG. 12 is a plan view of the clip used in the embodiment of FIG. 11;

FIG. 13 is a fragmentary cross-sectional view illustrating the integral syringe barrel and cannula with a weakened cannula portion intermediate its length;

FIGS. 13A and 13B are cross-sectional views similar to FIG. 13 illustrating further aspects of weakened cannula portions to facilitate break-off of the distal portion of the cannula;

FIG. 14 is a view similar to FIG. 13 illustrating the application of a standard Luer fit with metal needle to the remaining portion of the cannula; and

FIG. 14A is a view similar to FIG. 13 illustrating a Luer Lok™ for retaining the metal needle and the barrel of the syringe.

### BRIEF DESCRIPTION OF THE INVENTION

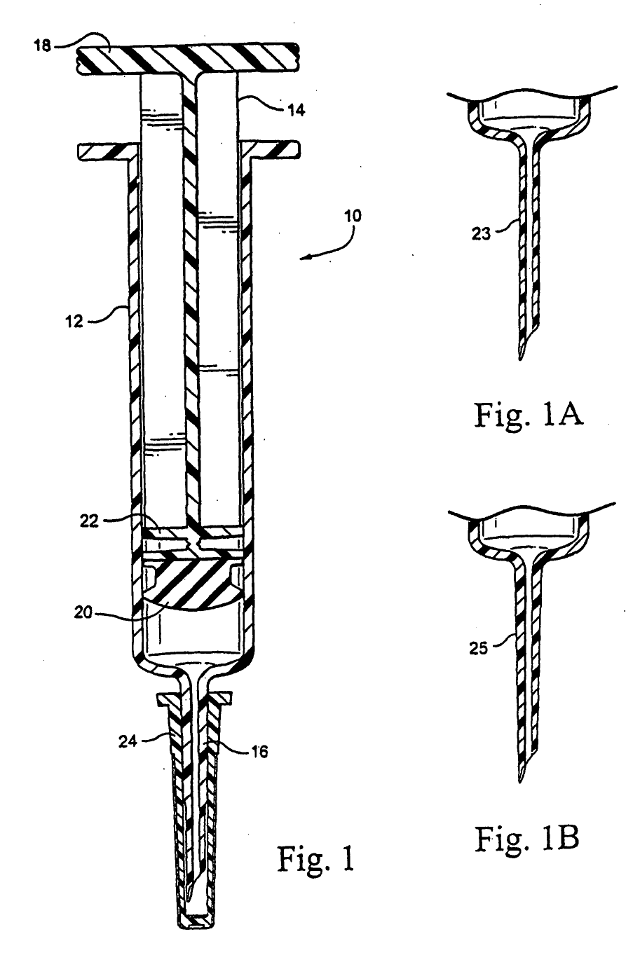

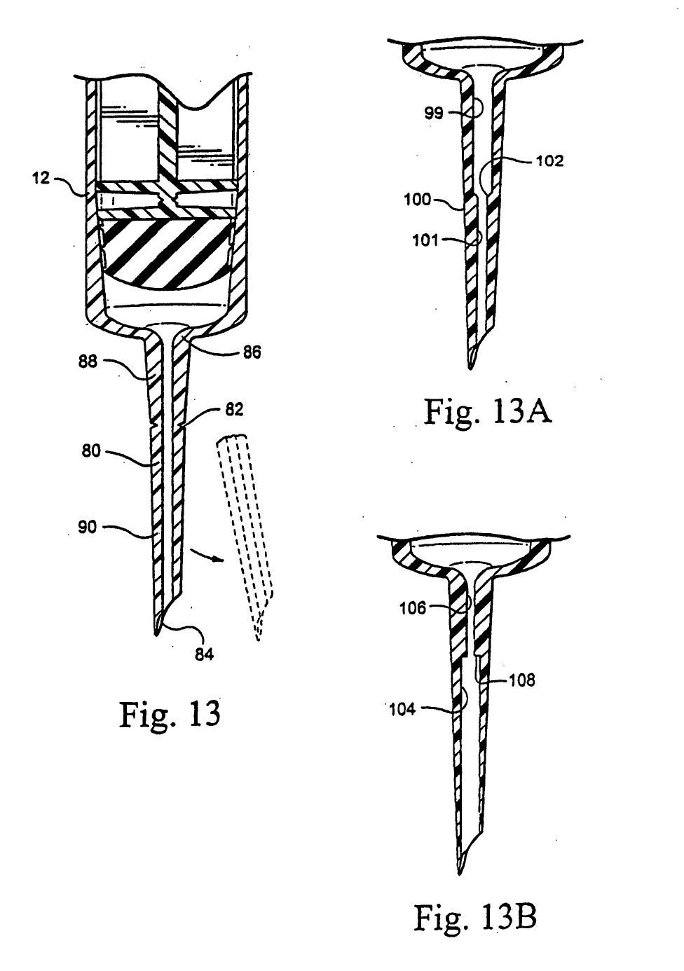

Referring to FIG. 1, there is illustrated a syringe generally designed 10 comprised of an elongated generally cylindrical barrel 12, a plunger 14 and a cannula 16. The plunger 14 includes an intermediate shank portion, preferably of cruciform cross-section having a thumb press 18 at a proximal end and an elastomeric bung 20 at its distal end. Between the cruciform shank and the bung 20 is a frangible section 22 described below. Additionally illustrated is a cap 24 overlying the cannula 16. This cap may be attached by a press fit, threaded connection or other means to the barrel or alternatively any other attachment to or part of the barrel such as a Luer Lok threaded cylinder. The cap may contain a plug of elastomer to steady or seal the end of the cannula. There may also be openings at both ends of the cap enabling it to be slid over the cannula and act as a lever for snapping off the portions of the cannula distal to the Luer dimensional area as shown in FIG. 13.

As illustrated, the barrel 12 and the cannula 16 are molded as a single unitary piece thereby eliminating differences in materials between the barrel and cannula as well as any adhesive typically required to connect a metal needle to the barrel. The plastic parts of the syringe including the integral barrel and cannula are formed of a plastic material and preferably a Cyclic Olefin Copolymer (COC) which acts as a barrier to water or other molecules thereby allowing the syringe to be prefilled with water or other solutions or dry and/or powdered medication. The syringe 10 of course operates in the usual manner whereby axial withdrawal of the plunger relative to the syringe barrel may draw fluid into the syringe and axial displacement of the plunger toward the distal end of the barrel expels fluid from the syringe.

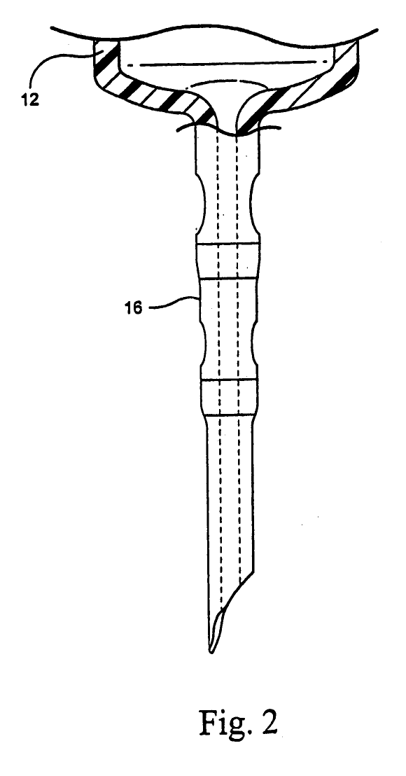

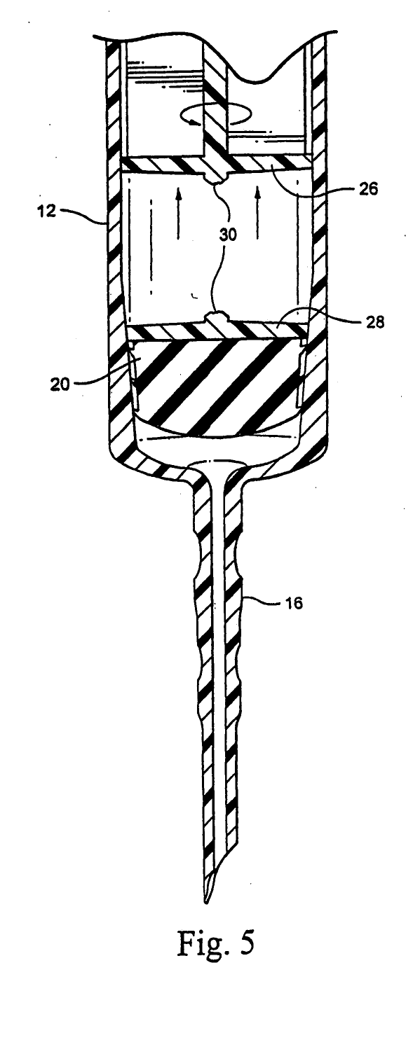

The shape of the integral cannula 16 may be the same as disclosed in U.S. Pat. No. 6,616,632 incorporated herein by reference and it will be appreciated that the attributes thereof are applicable to the present invention. Also, the cannula outer surface may have formations which provide respective different resistances to movement of the cannula through a membrane. The formations may be of the type described and illustrated with respect to FIGS. 1-6 of U.S. Pat. No. 6,616,632. For example, the cannula may penetrate unsupported membranes easily, i.e., the top of a medication vial, but will penetrate skin only with the greatest difficulty. FIG. 1 illustrates a cannula having a stepped outer surface. FIG. 1A illustrates a cannula integral with the barrel having a smooth, generally cylindrical outer surface 23 throughout its length. FIG. 1B illustrates a cannula integral with the barrel having a smooth continuous tapered outer surface 25.

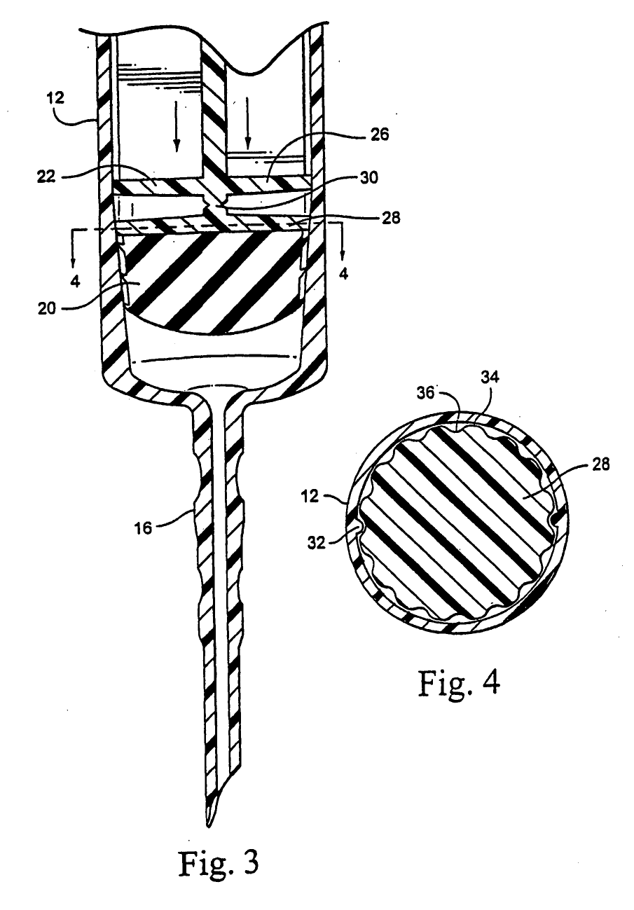

Referring to FIGS. 3-5, the frangible section 22 includes a pair of axially spaced upper and lower plates 26 and 28, respectively, joined one to the other by an axial reduced diameter stem or connector 30 which enables the upper plate 26, shank and proximal end of the plunger to be separated from the lower plate 28 and bung 20 in response to rotation of the plunger about the syringe axis and relative to the barrel. To accomplish this, the interior surface of the barrel adjacent the distal end is provided with one or a plurality of radially inwardly projecting ribs 32, two such ribs being illustrated in FIG. 4 at diametrically opposite locations. The lower plate 28 has a periphery, e.g., formed by a plurality of radial outward projections 34 and adjacent recesses 36. It will be appreciated that upon axial displacement of the plunger toward the distal end of the barrel, one or more of the recesses 36 will be engaged by one or more of the ribs 32 of the barrel permitting axial movement of the plunger relative to the barrel but minimizing or eliminating relative rotational movement of the plunger and barrel. The rib and groove arrangement, however, need not be diametrically opposite since the rib and groove arrangements may be asymmetrical relative to one another to preclude jamming and the chance of axial dead-on engagement is significantly reduced. A number of variations on the internal surface of the barrel involving ribs, grooves or flats with corresponding features on the plunger are possible to effect rotational fixation between the distal portion of the plunger and the distal end of the barrel. It will be appreciated that reuse of the syringe can then be prevented by rotating the plunger relative to the barrel breaking the stem or connection 30 of the frangible section 22. The cooperation between the one or more ribs 32 and recesses 36 of barrel 12 and lower plate 28 prevents relative rotation of lower plate 28 and barrel 12 upon rotation of the plunger, relative to barrel 12. It will also be appreciated that the upper plate 26 is not necessary although desirable for structural reasons and that features other than ribs and grooves can be used to achieve the desired rotational fixation.

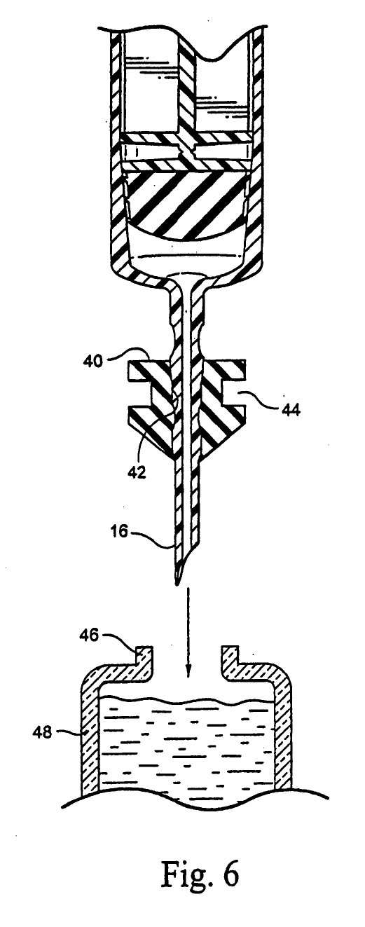

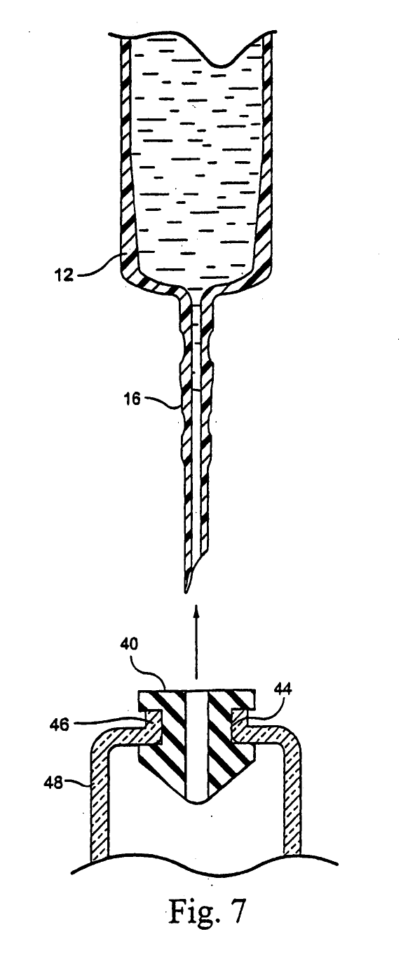

Referring to FIGS. 6 and 7, the cannula 16 may be provided with a collar 40 formed of an elastomeric material rendering the collar flexible, resilient and in essence spongy. The elastomeric material may be open cell or closed cell and has a central opening 42 enabling the collar to fit about the cannula 16. The collar 40 also includes an annular groove 44 sized to receive the lip 46 of a standard vial 48 or tapered to fit a plurality of sizes of vial. When it is desired to withdraw liquid from vial 48, cannula 16 is inserted into the vial 48 and collar 40 engages the lip of the vial with the lip 46 being received in the groove 44 as illustrated in FIG. 7. The taper of the collar facilitates insertion of the collar into the vial lip 46. It will be appreciated that when the cannula is disposed in vial 48 and lip 46 engages collar 40, the collar seals between the cannula and the collar and between the collar and the lip. Thus, the vial can be inverted to allow easy access to all of the fluid contents using a cannula which is relatively short relative to the length of the vial. The collar may be designed so that when the cannula is removed from the vial, the collar remains in the neck of the glass vial rendering the same cannula not easily reused and certainly not reusable with the original collar 40. The collar may be provided without a groove 44 in those instances where the collar may be retained on the cannula 16 subsequent to removing the cannula from the vial.

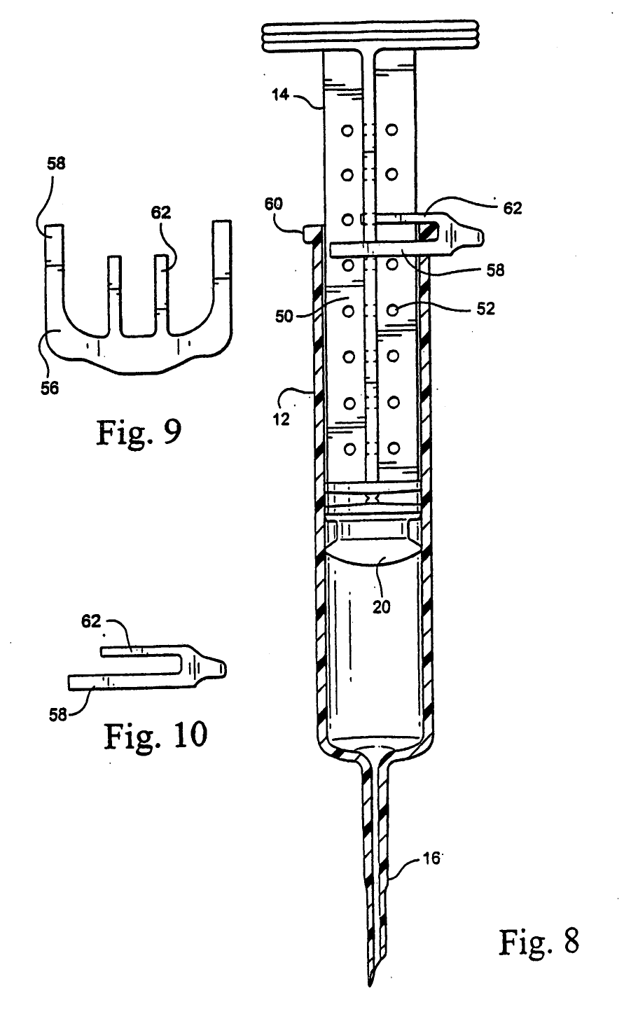

Referring to FIGS. 8-10, the plunger may be fixed at selected axial positions relative to the barrel. This prevents relative movement between the plunger and the barrel during transportation of the syringe, during filling using this mechanism and enables standardization of the syringe size so that the syringe can be filled to selective levels with selected quantities of fluid. To accomplish this in one embodiment hereof, the cruciform intermediate portion of the plunger 14 is provided in each of its wings 50, with a plurality of axially spaced openings 52. The openings are indexed relative to the proximal end of the barrel which correlates with a predetermined quantity of fluid within the barrel between its distal end and the bung 20.

Referring to FIGS. 9 and 10, a stabilization clip 56 includes a pair of outer legs 58 for reception below and engagement with the finger press 60 of the proximal end of barrel 12 and a pair of inner legs 62 for reception in the selected pairs of opening 52. Thus by withdrawing the plunger relative to the barrel such that a selected quantity of fluid resides in the distal end of the barrel, the clip may be applied to the barrel with the outer legs 58 underlying and engaging finger press 60 and the inner legs 62 received in the selected pair of openings 52 locking the plunger and barrel to one another against axial movement. This locking action also ensures that during handling of the syringe prior to removal of the clip 56, the contents of the syringe are not inadvertently expressed prematurely. Upon removal of the stabilization clip 56, the syringe can be used in the normal conventional fashion. Clip 56 can be formed integrally with barrel 12.

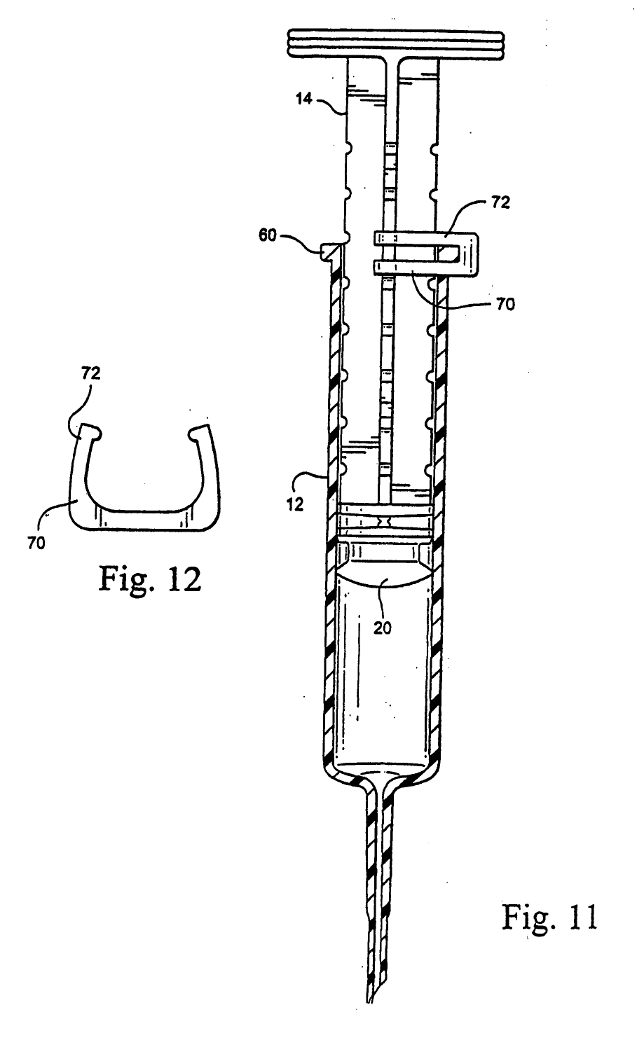

Referring to FIGS. 11 and 12, there is illustrated another embodiment by which the plunger and barrel are fixed to one another at selected axial locations. In this form, the margins of the cruciform wings of the plunger 14 have recesses or grooves at axially spaced locations therealong. A single U-shaped clip 70 having axially spaced pairs of outer legs 72 is provided. In use, the axially spaced pairs of outer legs 72 may straddle the finger press 60 with the upper pair of legs 72 spring biased inwardly to engage a selected pair of grooves along the margins of the plunger to fix the plunger at selected axial positions relative to the barrel. To use the syringe, clip 70 is removed from engagement with the plunger enabling the plunger for axial movement relative to the barrel.

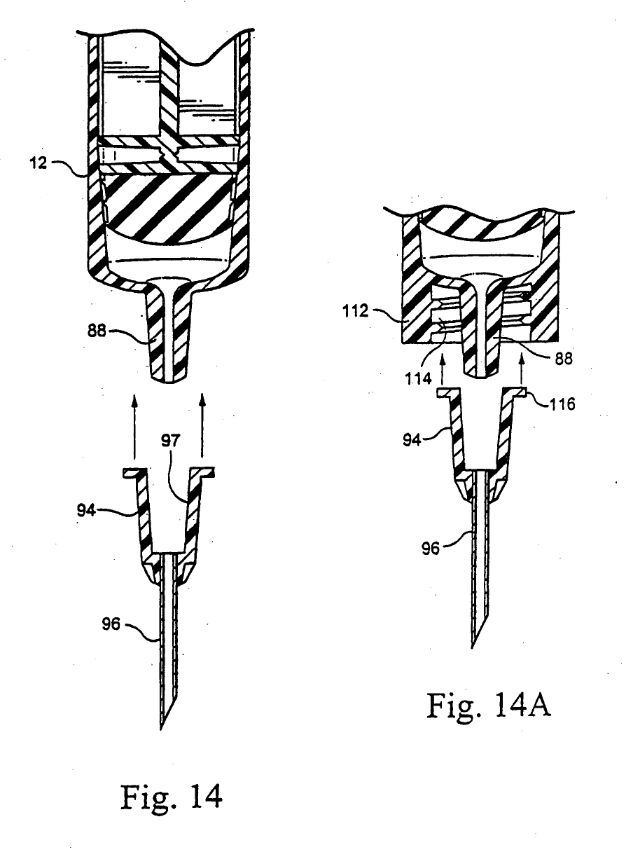

Referring now to FIGS. 13 and 14, the syringe barrel 12 may be provided with an integrally formed cannula 80 similarly as in the prior embodiment. However, in this aspect, the cannula 80 has a weakened portion 82 intermediate the distal end 84 and the base 86 of the cannula. The remaining portions of the syringe are similar as previously described. In this aspect, the proximal portion 88 of the cannula 80 is sized, dimensioned and configured to receive the standard Luer fit containing a sharp metal needle or mate with any Luer standard male fitting such as a Luer activated valve often used for access IV administration sets. That is, the distal portion 90 of the cannula can be broken from the proximal portion 88 at the weakened portion 82 leaving the proximal portion 88 sized, dimensioned and configured to receive the Luer fit. Referring to FIG. 14, the Luer fit comprises a hub 94 mounting a metal needle 96. The hub 94 includes an interior tapered female receptacle 97 for receiving the tapered male proximal portion 88 once the distal portion 90 of the cannula 80 has been snapped off. Thus the syringe of FIG. 13 can be converted after use, for example after withdrawing medication from a vial, as a standard syringe for injecting through an individual's skin utilizing a metal needle. The plunger break off feature previously described would prevent subsequent reuse of the syringe.

Referring back to FIGS. 13A and 13B, the outer cylindrical or tapered surfaces of the cannula may be smooth and continuous while at the same time provide a weakened portion intermediate the length of the cannula enabling the cannula tip to be broken off similarly as previously described. In FIG. 13A, the internal diameters 99 and 101 of the cannula 100 are different in transition at a weakened point 102, facilitating break-off of the tip of the cannula. In FIG. 13B, the larger diameter portion 104 of the cannula transitions to a smaller diameter portion 106, thus forming a weakened portion 108 at the transition which facilitates break-off the cannula tip leaving the generally tapered proximal portion of the cannula sized, dimensioned and configured to receive the Luer hub 96.

In FIG. 14A, the proximal portion 88 of the syringe barrel left after the distal portion of the cannula has been broken from the proximal portion may receive the hub 94 mounting the metal needle 96. In this aspect, however, the barrel has a cylindrical extension 112 internally threaded at 114 enabling the flanges 116 of the standard Luer Lok™ and metal needle to thread onto the barrel with the proximal portion 88 within the hub. It can be appreciated that the Luer Lok feature may be molded integral to the barrel as shown but equally may be a separate component which may be itself threaded onto external threads molded on the barrel or cooperate and be affixed by other features such as a snap fit with anti-rotational features or other types of connector mechanism present on the external surface of the distal barrel and or proximal cannula 94. Thus, the integral barrel and cannula may be utilized with either a Luer fit or Luer Lok™ arrangement once the distal end of the cannula is broken off from the cannula leaving the proximal cannula portion 88 to receive the hub of the needle.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed:

1. A syringe comprising:
a syringe barrel having proximal and distal ends;
a plunger in part disposed in said barrel and having a bung adjacent an end of the plunger within the barrel, said plunger being movable toward and away from the distal end of the barrel;
the distal end of the barrel terminating in an elongated end cannula having a central opening in communication with the interior of the barrel between the distal end of the barrel and plunger bung for receiving fluid into or expelling fluid from the barrel;
the barrel and cannula being molded from and formed of a single unitary plastic material.

2. A syringe according to claim 1 wherein said material is a cyclic olefin copolymer.

3. A syringe according to claim 1 wherein said cannula includes formations along the outer surface thereof affording respective differential resistances to movement of the cannula through a membrane.

4. A syringe according to claim 1 wherein the plunger has an end portion adjacent the bung and separable from remaining portions of the plunger, said plunger end portion and the barrel having cooperating surfaces enabling separation of the plunger end portion and the remaining portions of the plunger in response to relative rotation of the plunger and the barrel.

5. A syringe according to claim 4 wherein said cooperating surfaces are engageable with one another in response to axial displacement of the plunger toward the distal end of the barrel.

6. A syringe according to claim 5, wherein said surfaces include at least one inwardly extending rib carried by the barrel adjacent the distal end thereof and at least one projection carried by the plunger end portion which, upon engagement, preclude relative rotation of the plunger end portion and barrel.

7. A syringe according to claim 1 including an annular collar about the cannula, said collar having an annular groove about an external surface thereof sized to receive the tip of a medication vial enabling the collar to seal between the cannula and the vial lip.

8. A syringe according to claim 7 wherein said collar is formed of a flexible resilient closed or open cell material.

9. A syringe according to claim 7 wherein the collar and cannula are releasably secured to one another enabling the cannula to be withdrawn from the collar leaving the collar about the vial lip.

10. A syringe according to claim 1 including a clip engageable with the plunger and the barrel at each of a plurality of selected axial locations of the plunger relative to the barrel thereby to fix the axial location of the plunger relative to the barrel at a selected location therealong.

11. A syringe according to claim 10 wherein the barrel includes a finger press at said proximal end thereof, said clip being engagable with said finger press and with an opening in the plunger or a recess formed along the plunger.

12. A syringe according to claim 1 wherein said cannula includes formations along the outer surface thereof affording respective differential resistances to movement of the cannula through a membrane, wherein the plunger has an end portion adjacent the bung and separable from remaining portions of the plunger, said plunger end portion and the barrel having cooperating surfaces enabling separation of the plunger and portion and the remaining portions of the plunger in response to relative rotation of the plunger and the barrel.
said plunger end portion and the barrel having cooperating surfaces enabling separation of the plunger end portion and the remaining portions of the plunger in response to relative rotation of the plunger and the barrel.

13. A syringe according to claim 1, wherein the plunger has an end portion adjacent the bung and separable from remaining portions of the plunger, said plunger end portion and the barrel having cooperating surfaces enabling separation of the plunger end portion and the remaining portions of the plunger in response to relative rotation of the plunger and the barrel, and an annular collar about the cannula, said collar having an annular groove about an external surface thereof sized to receive the tip of a medication vial enabling the collar to seal between the cannula and the vial lip.

14. A syringe according to claim 1 wherein the plunger has an end portion adjacent the bung and separable from remaining portions of the plunger, said plunger end portion and the barrel having cooperating surfaces enabling separation of the plunger end portion and the remaining portions of the plunger in response to relative rotation of the plunger and the barrel, and a clip engageable with the plunger and the barrel at each of a plurality of selected axial locations of the plunger relative to the barrel thereby to fix the axial location of the plunger relative to the barrel at a selected location therealong.

15. A syringe comprising:
a syringe barrel having proximal and distal ends;
a plunger in part disposed in said barrel and having a bung adjacent an end of the plunger within the barrel, said plunger being movable toward and away from the distal end of the barrel;
the distal end of the barrel terminating in an elongated end cannula having a central opening in communication with the interior of the barrel between the distal end of the barrel and the plunger bung, for receiving fluid into or expelling fluid from the barrel;
the plunger having an end portion adjacent the bung and separable from remaining portions of the plunger, said plunger end portion and the barrel having cooperating surfaces enabling separation of the plunger end portion and the remaining portions of the plunger in response to relative rotation of the plunger and the barrel.

16. A syringe according to claim 15 wherein said cooperating surfaces are engageable with one another in response to axial displacement of the plunger toward the distal end of the barrel.

17. A syringe according to claim 16 wherein said surfaces include at least one inwardly, extending rib carried by the barrel adjacent the distal end thereof and at least one projection carried by the plunger end portion which, upon engagement, preclude relative rotation of the plunger end portion and barrel.

18. A syringe comprising:
a syringe barrel having proximal and distal ends;
a plunger in part disposed in said barrel and having a bung adjacent an end of the plunger within the barrel, said plunger being movable toward and away from the distal end of the barrel;
the distal end of the barrel terminating in an elongated end cannula having a central opening in communication with the interior of the barrel between the distal end of the barrel and plunger bung for receiving fluid into or expelling fluid from the barrel; and
an annular collar about the cannula, said collar having an annular groove about an external surface thereof sized to receive the tip of a medication vial enabling the collar to seal between the cannula and the vial lip.

19. A syringe according to claim 18, wherein said collar is formed of a flexible, resilient closed or open cell material.

20. A syringe according to claim 18, wherein the collar and cannula are releasably secured to one another enabling the cannula to be withdrawn from the collar leaving the collar about the vial lip.

21. A syringe comprising:
a syringe barrel having proximal and distal ends;
a plunger in part disposed in said barrel and having a bung adjacent an end of the plunger within the barrel, said plunger being movable toward and away from the distal end of the barrel;
the distal end of the barrel terminating in an elongated end cannula having a central opening in communication with the interior of the barrel between the distal end of the barrel and plunger bung for receiving fluid into or expelling fluid from the barrel; and
including a clip engageable with the plunger and the barrel at each of a plurality of selected axial locations of the plunger relative to the barrel thereby to fix the axial location of the plunger relative to the barrel at a selected location therealong.

22. A syringe according to claim 21, including a clip engageable with the plunger and the barrel at each of a plurality of selected axial locations of the plunger relative to the barrel thereby to fix the axial location of the plunger relative to the barrel at a selected location therealong.
